(19) United States
(12) Patent Application Publication
(10) Pub. No.: US 2014/0256832 A1
(43) Pub. Date: Sep. 11, 2014

(54) FULLERENE-DERIVED CELLULOSE NANOCRYSTAL, THEIR PREPARATION AND USES THEREOF

(71) Applicant: CELLUFORCE INC., Montreal (CA)

(72) Inventors: Zhaoling Yao, Waterloo (CA); Janelle Huimin Tam, Waterloo (CA)

(73) Assignee: CELLUFORCE INC., Montreal (CA)

(21) Appl. No.: 14/198,999

(22) Filed: Mar. 6, 2014

Related U.S. Application Data
(60) Provisional application No. 61/773,245, filed on Mar. 6, 2013.

(51) Int. Cl. C08B 15/00; A23L 3/3544; A61K 47/38; A61K 8/73; C09K 15/06; C08B 37/16

(52) U.S. Cl. CPC C08B 15/00; C09K 15/06; C08B 37/0012; A61K 47/38; A61K 8/738; A61K 8/731; A23L 3/3544
USPC 514/781; 252/407; 536/99; 426/321

(57) ABSTRACT

The disclosure relates to a fullerene-derived cellulose nanocrystals, process for preparing same and methods of using said nanocrystals.

### CROSS-REFERENCE TO RELATED APPLICATIONS

This application claims priority to U.S. Provisional Application No. 61/773,245 filed Mar. 6, 2013, the entire contents of which is specifically incorporated by reference herein without disclaimer.

### BACKGROUND OF THE DISCLOSURE

1. Field of the Invention

The disclosure relates to a fullerene-derived cellulose nanocrystals, process for preparing same and methods of using said nanocrystals.

2. Description of Related Art

Cellulose nanocrystal (CNC), discovered in 1949 by Bengt Ranby, was prepared from acid hydrolysis of naturally existing cellulose semicrystals. It is abundant, renewable and biodegradable, CNC can be used as a building block for the preparation of various functional nano-materials as it possesses a number of advantages, such as low density, high specific surface area, and superior mechanical properties. The numerous hydroxyl groups on the nanocrystal surface can be used to modify CNC.

Fullerene (C₆₀), which was discovered in 1985 by Kroto, Curl and Smalley possesses a diverse range of attractive properties, such as electronic, conducting, antioxidant and magnetic properties due to its unusual symmetry and electron conjugate characteristics. However, its strong cohesive nature and poor solubility in common aqueous and organic solvents has hampered its applications in biomedical science Therefore, various types of functionalization strategies have been explored to broaden and expand its end-use applications. C₆₀-polymer systems have been developed and their properties elucidated to allow for their applications in a wide range of fields, such as diagnostics, pharmaceuticals, environmental and energy.

The biological application of fullerenes was reviewed by Bakery and co-workers (Bakry, R., et al Int J Nanomedicine, 2, 639-649 (2007)). Based on the findings that C₆₀ encapsulated within polyvinylpyrrolidone (PVP) is water-soluble (Yamakoshi, Y. N., et al J. Chem. Soc., Chem. Comm., 517-518 (1994)), and C₆₀ exhibits free radical scavenging activity (Krusic, P. J., et al. J. Am. Chem. Soc., 113, 6274 (1991)), the antioxidant and anti-uv abilities in human skin keratinocytes using PVP encapsulated C₆₀ were investigated (Xiao, L. et al. Bioorganic & Medicinal Chemistry Letters, 16, 1590-1595 (2006) and Xiao, L. et al. Biomedicine & Pharmacotherapy, 59, 351-358 (2005)). A commercial product that can slow down the aging of skin by Vitamin C₆₀ BioResearch Corp was registered for cosmetic application and subject of a patent application in US 2010/0015083.

### SUMMARY OF THE DISCLOSURE

In one aspect, there is provided a process for functionalizing cellulose nanocrystal comprising contacting a soluble fullerene or fullerene derivative or complex thereof, CNC and a free radical generator capable of generating radicals on said CNC and isolating said functionalized cellulose nanocrystal.

In one aspect, there is provided a method for improving resistance to free radical degradation of a material, optionally an organic material, comprising adding to said material a fullerene-derived cellulose nanocrystal.

In one aspect, there is provided a method for scavenging free radical in a substance comprising contacting said substance with a fullerene-derived cellulose nanocrystal.

In one aspect, there is provided a fullerene-cellulose nanocrystal derivative, wherein said cellulose nanocrystal is covalently bonded to one or more fullerene or fullerene derivative or soluble complex thereof.

### BRIEF DESCRIPTION OF THE FIGURES

FIG. 1 is a schematic representation of a process to form CNC-fullerene-(cyclodextrin);

FIG. 2 is a bar graph of antioxidant activity evaluated by the rate constant of CNC and CNC-C₆₀-(β-cyclodextrin);

FIG. 3 is a bar graph the analysis showing anti-oxidant properties of fullerene-derivatives.

### DESCRIPTION OF THE EMBODIMENTS

CNC used in this study was prepared from the acid hydrolysis of wood fibers using sulfuric acid, leaving behind small fractions of carboxylic and sulfate ester groups on the surface of CNC. The negatively charged sulfate groups allow the CNC to disperse well in water. The dimensions of CNC are about 5-20 nm in diameter and 100-200 nm in length.

It has been shown that persulfate salt is efficient in converting hydroxyl groups to radicals (Roy, D., Semsarilar, M., Guthriea, J. T., Perrier, S., Cellulose modification by polymer grafting: a review, Chem. Soc. Rev., 38, 2046-2064 (2009))

The poor solubility of fullerenes such as C₆₀ in aqueous solvent requires the modification of fullerene in order to attach fullerene onto the surface of CNC in aqueous solution. A stable inclusion complex of β-cyclodextrin with C₆₀ was reported (Murthy, C. N., Geckeler, K. E., The water-soluble β-cyclodextrin-C₆₀ fullerene complex, Chem. Commun., 1194-1195 (2001)) and the complex also showed radical scavenging capability.

As used herein, “fullerene” means any fullerene or a derivative thereof, preferably a fullerene having a radical scavenging ability. Fullerenes include C₂₀ to C₁₀₀ and particularly C₆₀ and C₇₀ and there mixtures thereof. The fullerene also include those in which more than one of them are bound through a chain such as an alkylene chain. The preparation and modification of cyclodextrins is well known and within the skills of the artisan without undue burden.

As used herein, “cyclodextrin” is meant to comprise those cyclodextrins that can form a host-guest complex with one member of the fullerene used herein. Cyclodextrins may have different sizes and/or be modified to form the most desirable host-guest complex depending on the fullerene. The modified cyclodextrins may also allow to modulate their physicochemical properties (such as solubility). The preparation and modification of cyclodextrins is well known and within the skills of the artisan without undue burden. Cyclodextrins may have 5 or more 1->4 linked α-D-glucopyranoside units. Typical cyclodextrins contain a number of glucose monomers ranging from six to eight units in a ring, creating a cone shape. For example: α-cyclodextrin: six membered molecule; β-cyclodextrin: seven membered molecule and γ-cyclodextrin: eight membered ring molecule.

As used herein, “free radical generator” intends to refer to reagents that are capable of generating radicals on CNC. Sodium persulfate was used in the below examples however other conditions may be suitable. For example, see Roy, D., et al (Supra) which discusses other conditions that may to generate radicals on CNC, such as using Fenton's reagent, oxidation via Ce(IV) ions, radiation-induced radical formation, etc.

Various conditions may be suitable to bring the hydrophobic fullerene molecule into water or aqueous solutions. The approach used in the examples below was selected from the formation of host-guest complexes using β-cyclodextrin. A skilled person would also immediately understand from the present disclosure that γ-cyclodextrin [Yoshida, Z.-i. et al. Angew. Chem., Int. Ed. Engl. 1994, 33, 1597-1599] or calixarenes [R. M. Williams et al. Recl. Tray. Chim. Pays-Bas,1992, 111, 531] can also be used to form water-soluble fullerene complexes.

### General Preparation of CNC-Fullerene-(cyclodextrin)

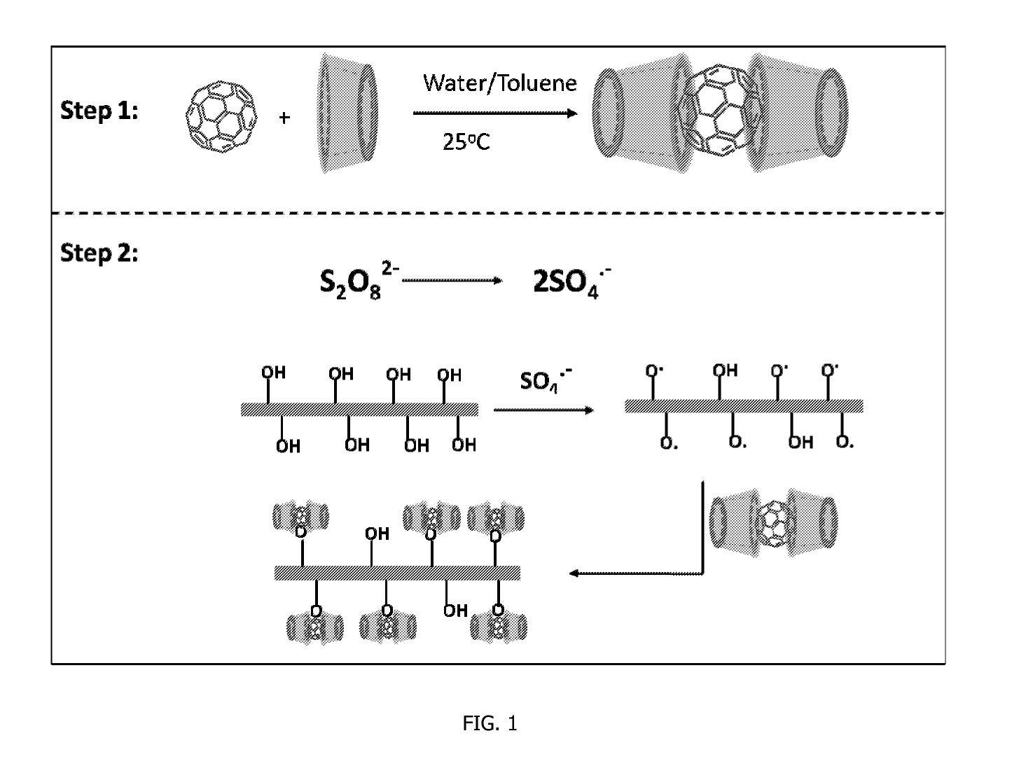

The reaction to form CNC-fullerene-(cyclodextrin) involved two steps (FIG. 1). First, water-soluble fullerene-(cyclodextrin) complex was prepared via a solvent exchange technique. Then, the radical coupling reaction was performed by converting the hydroxyl groups on CNC to free radicals in the presence of a suitable “free radical generator”, and these free radicals were captured by the fullerene-(cyclodextrin) complex to form CNC-fullerene-(cyclodextrin).

### EXAMPLE 1

### Synthesis of C₆₀ Methyl-Beta-Cyclodextrin Complex

Saturated solutions of C₆₀ in toluene and β-CD in water were vigorously mixed at 25° C. for two weeks, so that the C₆₀ molecules were encapsulated by β-CD to yield a soluble β-CD-C₆₀ complex. The product was dialyzed using dialysis membrane tubing with 3500 Molecular cut-off for two days to remove the excess β-CD.

### EXAMPLE 2

### Synthesis of CNC-C₆₀-(Methyl-Beta-Cyclodextrine)

26 mg (0.11 mmol) sodium persulfate and 20 mg CNC were dissolved in 10 mL C₆₀-(β-cyclodextrin) solution in a reaction flask. The mixture was bubbled with argon for 30 mins and stirred at 65° C. for 24 h. The final reaction mixture was purified by dialysis for two days in a dialysis tubing with MW cut off of 12-14K. Finally fluffy yellow product was isolated after freeze drying.

Both examples 1 and 2 produced homogenous, brown colored solutions even after dialysis, when unreacted cyclodextrin in example 1 and fullerenes in example 2 were removed, confirming that fullerenes (responsible for the brown color) were present in both complexes. A UV spectrum obtained on the products of the examples showed a peak at 350 nm, the characteristic absorption line of fullerenes, as evidence for the success of the grafting reaction.

It will be clear to the skilled person that the number of fullerenes attached to CNC depends on the ratio of fullerene-CD and CNC used for the reaction. The ratio of fullerenes on CNC for the final complex can be determined using a UV-vis by measuring the extinction coefficient of fullerene-CD and the peak intensity around 350 nm of CNC-fullerenes-CD in aqueous solution.

### EXAMPLE 3

### Antioxidant Measurements of CNC and CNC-C₆₀-(β-Cyclodextrin)

The radical scavenging or antioxidant property of CNC and CNC-fullerene compounds using the stable free radical, 2,2-diphenyl-1-picrylhydrazyl (DPPH) were evaluated. This process was monitored by UV-Vis spectroscopy, which showed a reduction in the peak absorbance at 517 nm when CNC or CNC-fullerene compounds were mixed with DPPH solution.

Fresh stock solutions of DPPH in alcohol reagent at concentrations of 1 mg/mL were prepared daily. Control experiments were performed with ascorbic acid. The results matched literature data (Nenadis, N., et al. Journal of American Oil Chemists' Society 79.12 (2002): 1191-1195. Print). A DPPH calibration curve determined experimentally agreed with published results (Foti, M. C. et al Journal of Organic Chemistry 69 (2004): 2309-2314. Print.), confirming that the DPPH samples had not deteriorated. Solvent control experiments were run to ensure that the solvent used did not affect the results.

In this measurement, an aqueous solution with 1 wt % of CNC or CNC-C₆₀-(β-cyclodextrin) were first prepared and 2.5 mM DPPH solutions were prepared in alcohol reagent (containing anhydrous ethyl alcohol 90%±1% v/v; methyl alcohol approx. 5% v/v; 2-propanol approx. 5% v/v). For each reaction, 0.15 mL DPPH solution was added to a mixture of 1 mL antioxidant solution (1 wt %) and 1.85 mL alcohol reagent in a cuvette, which was then mixed vigorously. The absorbance at 517 nm was then monitored over time. The antioxidant activity was evaluated by the rate constant, which was calculated using the following equation:

$$- kt = ln \frac{{A}_{∞} - {A}_{t}}{{A}_{∞} - {A}_{0}}$$

where t is time and A_{∞}, Aₜ, A₀ are the absorbances at t equals to infinity, time t and zero, respectively. The rate constant (k) provides information on the rate of reaction: the higher the k-value, the faster the reaction, and the higher the antioxidant properties of the material tested (provided the concentrations are constant).

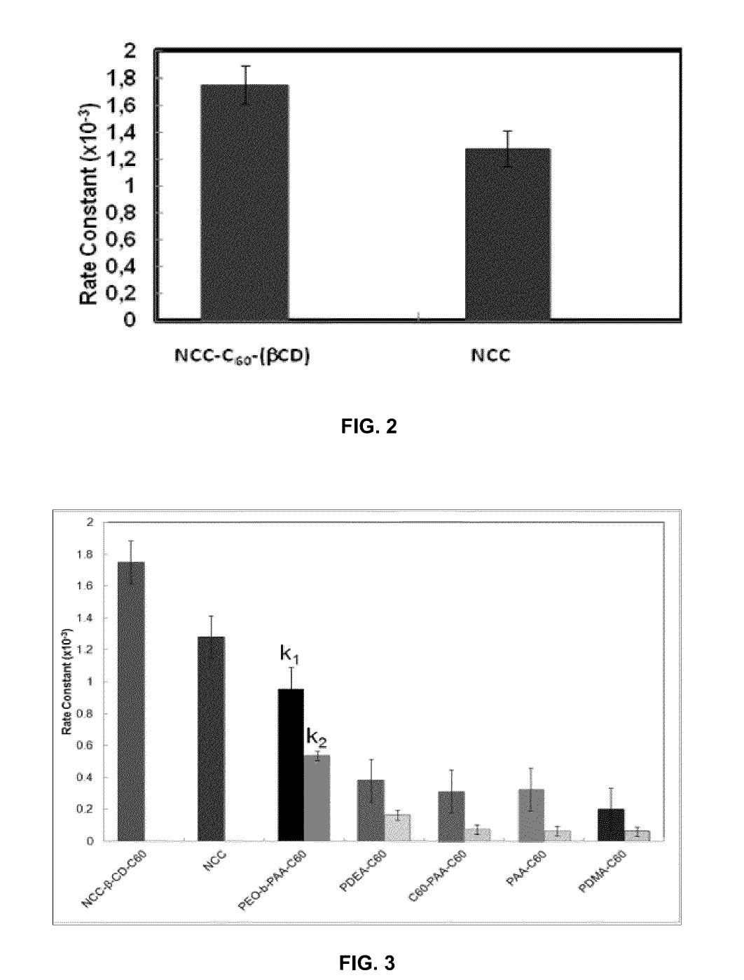

The rate constants for CNC and CNC-C₆₀-(β-cyclodextrin) are shown in FIG. 2, from which we can conclude that CNC-C₆₀-(β-cyclodextrin) had a faster antioxidant activity than CNC.

### EXAMPLE 4

### Comparison of Antioxidant Properties Between CNC-C₆₀-(β-Cyclodextrin and Other Polymer-Fullerene Derivatives

Using a protocol similar to example 3, various other antioxidant compounds were tested. The analysis shows that CNC-C₆₀-(β-cyclodextrin) compound possesses a better anti-oxidant properties than other fullerene-derivatives as shown in FIG. 3. In FIG. 3, k1 and k2 are the rate constants for the scavenging reaction of the free radicals by the unimeric fullerene-polymer and fullerene-polymer clusters/aggregates respectively.

Table 1 is summarizing the acronyms used in FIG. 3

| Acronym | Polymer-Fullerene |
|---|---|
| PAA- C₆₀ | Mono-fullerene end-capped poly(acrylic acid) |
| C₆₀-PAA-C₆₀ | Di-fullerene end-capped poly(acrylic acid) |
| PEO-b-PAA-C₆₀ | Poly(ethylene oxide)-block-poly(acrylic acid)- |
| | fullerene |
| PDMA-C₆₀ | Poly(2-(dimethylamino) ethyl methacrylate)- |
| | fullerene |
| PDEA-C₆₀ | Poly(2-diethylamino) ethyl methacrylate)-fullerene |

This suggests that the invention disclosed herein displays better performance than the fullerene-polymer systems developed earlier by Vitamin-C₆₀ for cosmetic applications.

In the above examples, water-soluble C₆₀-(β-cyclodextrin) was covalently attached to the surfaces of CNC via a radical coupling reaction. The antioxidant activity of CNC and CNC-fullerene-(cyclodextrin) measured by DPPH assay showed that these CNC-fullerene compounds can be used to scavenge radicals and such open the application as antioxidant ingredient in various applications.

The use of CNC instead of a synthetic polymer, such as PVP, will reduce the carbon footprint for this product.

The CNC-fullerene-(cyclodextrin) described herein can have a large scope of application such as in cosmeceutical, pharmaceutical and industrial applications. It is believed that the compounds can be used in substances to inhibit or retard oxidation.

The compounds described herein may be used as a food additive to maintain the quality of that food and to extend its shelf life.

The compounds may be added to industrial products as stabilizers in lubricants and polymers such as rubbers, plastics and adhesives to prevent the oxidative degradation and avoid a loss of strength and flexibility in these materials. The compounds may be incorporated in sealing materials or coating such as waxes used in protecting food products, fruits or vegetables.

As oxidative stress appears to be an important part of many human diseases, the compounds may also, for example, be added to a cosmetic or therapeutic composition in the form of an emulsion, a cream, a lotion, a facial mask, a cleansing agent, an ointment or a liquid dispersion. The composition may as such be for external use to prevent or treat skin aging and skin troubles caused by free radical damage. In addition, the product may be used in UV shielding composition.

While the invention has been described in connection with specific embodiments thereof, it is understood that it is capable of further modifications and that this application is intended to cover any variation, use, or adaptation of the invention following, in general, the principles of the invention and including such departures from the present disclosure that come within known, or customary practice within the art to which the invention pertains and as may be applied to the essential features hereinbefore set forth.

1. A process for functionalizing cellulose nanocrystal (CNC) comprising:
contacting
a) a soluble fullerene, a fullerene derivative or a complex thereof;
b) CNC; and
c) a free radical generator capable of generating radicals on said CNC; and
isolating the functionalized cellulose nanocrystal.

2. The process of claim 1, wherein said soluble fullerene or fullerene derivative or complex is a fullerene-(cyclodextrin) complex.

3. The process of claim 2, wherein said complex is a complex with a cyclodextrin or a derivative thereof comprising 5 or more 1->4 linked α-D-glucopyranoside units.

4. The process of claim 2, wherein said complex is a complex with α-cyclodextrin; β-cyclodextrin or γ-cyclodextrin.

5. The process of claim 1, wherein said fullerene is a C₂₀ to C₁₀₀ fullerene or a mixture thereof.

6. The process of claim 1, wherein said fullerene is a C₆₀ and C₇₀ fullerene or a mixture thereof.

7. The process of claim 1, wherein said functionalized CNC is a CNC-fullerene-(cyclodextrin) complex.

8. A fullerene-derived cellulose nanocrystal (CNC), wherein said CNC is covalently bonded to one or more fullerene or fullerene derivative or soluble complex thereof.

9. The fullerene-derived CNC of claim 8 which is a CNC-fullerene-(cyclodextrin) complex.

10. The fullerene-derived CNC of claim 8, wherein said fullerene or fullerene derivative or soluble complex thereof is a fullerene-(cyclodextrin) complex.

11. The fullerene-derived CNC of claim 10, wherein said complex is a complex with a cyclodextrin or a derivative thereof comprising 5 or more 1->4 linked α-D-glucopyranoside units.

12. The fullerene-derived CNC of claim 10, wherein said complex is a complex with α-cyclodextrin, β-cyclodextrin or γ-cyclodextrin.

13. The fullerene-derived CNC of claim 8, wherein said fullerene is a C₂₀ to C₁₀₀ fullerene or a mixture thereof.

14. The fullerene-derived CNC of claim 8, wherein said fullerene is a C₆₀ and C₇₀ fullerene or a mixture thereof.

15. The fullerene-derived CNC of claim 8 prepared by the process of claim 1.

16. A method for scavenging free radicals or for improving resistance to free radical degradation in a substance comprising contacting said substance with a fullerene-derived CNC as defined in claim 8.

17. The method of claim 16, wherein said substance is susceptible of undergoing oxidative degradation and is i) a cosmetic composition, ii) a pharmaceutical composition, iii) a food product, or iv) an industrial product.
